(19) United States
(12) Patent Application Publication
(10) Pub. No.: US 2025/0042844 A1
(43) Pub. Date: Feb. 6, 2025

(54) MULTI-IONIC SURFACTANTS

(71) Applicant: Ecolab USA Inc., St. Paul, MN (US)

(72) Inventors: Ashish Dhawan, Aurora, IL (US); Carter Martin Silvernail, Lakeville, MN (US)

(21) Appl. No.: 18/782,241

(22) Filed: Jul. 24, 2024

Related U.S. Application Data
(60) Provisional application No. 63/528,460, filed on Jul. 24, 2023.

(51) Int. Cl. C07C 233/89; C11D 1/88; C11D 1/90

(52) U.S. Cl. CPC C07C 233/89; C11D 1/883; C11D 1/90; C11D 2111/16

(57) ABSTRACT

Compounds, compositions and methods are provided as surfactants, fabric softening agents, corrosion inhibitors, bio-film inhibitors, biocides, and rheology modifiers. The multi-ionic surfactants are compounds having a structure corresponding to Formula 1, or a salt thereof:

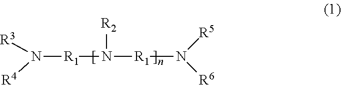

Wherein R¹ is C₁-C₁₀ alkylene; R² is independently hydrogen, —(CH₂)ₓ—NR²⁰R²¹, or —(CH₂)ₓ—C(R¹⁰)—C(O)—X—Z; R³ and R⁴ are independently hydrogen, —(CH₂)ₓ—C(R¹⁰)—C(O)—X—Z, or R³ and R⁴ together with the nitrogen they are attached to form a substituted nitrogen-containing heterocyclyl; R⁵ and R⁶ are independently hydrogen, —(CH₂)ₓ—C(R¹⁰)—C(O)—X—Z, or R⁵ and R⁶ together with the nitrogen they are attached to form a substituted nitrogen-containing heterocyclyl; R¹⁰ is hydrogen, alkyl, aryl, or alkaryl; R¹¹, R¹², and R¹³ are independently C₁ to C₁ alkyl or alkaryl; R²⁰ and R²¹ are independently hydrogen, —(CH₂)ₓ—NR²⁰R²¹, —(CH₂)ₓ—C(R¹⁰)—C(O)—X—Z, or together with the nitrogen they are attached to form a substituted nitrogen-containing heterocyclyl; X is NH or O; Z is hydrogen, L¹-NR¹¹R¹²R¹³, L²-PR¹¹R¹²R¹³, L³-COOH, L⁴-SO₃H, L⁵-PO₃H, or a salt thereof; L¹, L², L³, L⁴, and L⁵ are independently C₁-C₁ alkylene or alkenylene; n is an integer of 1 to 10; and x is an integer of 1 to 10; wherein at least one of R³ and R⁴, or R⁵ and R⁶ together form a substituted nitrogen-containing heterocyclyl; and wherein at least one R² is —(CH₂)ₓ—C(R¹⁰)—C(O)—X—Z or —(CH₂)ₓ—NR²⁰R²¹ wherein one of R²⁰ or R²¹ is —(CH₂)ₓ—C(R¹⁰)—C(O)—X—Z.

### FIELD OF THE INVENTION

Multi-ionic surfactants are provided as compounds, as compositions and in methods for use as surfactants, fabric softening agents, corrosion inhibitors, bio-film inhibitors, biocides, and rheology modifiers. The multi-ionic surfactant compound having a structure corresponding to Formula 1, or a salt thereof, is disclosed.

### BACKGROUND OF THE INVENTION

Corrosion of metal surfaces in aqueous media has long been a problem for industries such as the oil and gas industry, food/beverage industry, wash/sanitizing industry, pulp and paper, power generation, manufacturing, and utilities. For example, it is well known that during the production of oil and gas several other corrosive components are present such as brines, organic acids, carbon dioxide, hydrogen sulfide, and microorganisms. These aggressive constituents can cause severe corrosion as evidenced by surface pitting, embrittlement, and general loss of metal. The metallic surfaces can be composed of high alloy steels including chrome steels, ferritic alloy steels, austenitic stainless steels, precipitation-hardened stainless steels, and high nickel content steels, copper, and carbon steels.

In the food/beverage and wash/sanitizing industry, solutions such as sodium hypochlorite solutions are commonly used and are highly effective as bleaches and sanitizers for cleaning a variety of surfaces. However, sodium hypochlorite solutions are corrosive to many treated surfaces, in particular, metal surfaces become highly corroded.

There are several mechanisms responsible for corrosion of metals. In corrosive water systems, the overall corrosion rate is controlled by the reduction of oxygen inhibiting the cathodic reaction. However, the most robust and cost effective water treatment programs include both anodic and cathodic inhibitors to block reactions at both the anode and the cathode.

Corrosion inhibitors are usually surface-active compounds that form protective coatings on the surface of metals and suppress corrosion by preventing or reducing contact of the corrosive species to the pipeline surface. Common corrosion inhibitors are composed of amines, condensation products of fatty acids with polyamines, imidazolines, and/or quaternary ammonium compounds. Among the most frequently used corrosion inhibitors in crude oil and natural gas extraction are imidazoline derivatives and benzyldimethylalkylammonium chlorides.

Certain surfactants can be used as corrosion inhibitors and also have use in institutional (including FSR, HHC, & professional products), food and beverage, health care, quick service restaurants, pest elimination, textile care/laundry, water paper, mining, sensors, energy services, and consumer markets industries.

A surfactant compound usually contains a hydrophilic head and hydrophobic tail. Because of its unique structure, a surfactant has various applications in different fields. Although structures of surfactants are diverse and numerous, the existing surfactants can be classified into two general categories. One is conventional surfactants, and another is gemini surfactants. A conventional surfactant usually has a hydrophobic tail and a hydrophilic head. Depending on the characteristics of the hydrophilic head, a conventional surfactant can be one of nonionic, anionic, cationic, amphoteric, or zwitterionic surfactants. A gemini surfactant, on the other hand, has two hydrophobic tails and two hydrophilic heads. As the understanding of the relationship between a surfactant compound's structure and its function and mode of operation improves, the need for surfactant compounds having new or improved properties is increasing. The multi-ionic surfactant compositions described herein comprise multiple hydrophilic heads and multiple hydrophobic tails.

### BRIEF SUMMARY OF THE INVENTION

Disclosed herein are multi-ionic surfactant compounds, the multi-ionic surfactant compounds corresponding to the structure of Formula 1, or a salt thereof:

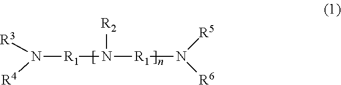

wherein R¹ is C₁-C₁₀ alkylene; R² is independently hydrogen, —(CH₂)ₓ—NR²⁰R²¹, or —(CH₂)ₓ—C(R¹⁰)—C(O)—X—Z; R³ and R⁴ are independently hydrogen, —(CH₂)ₓ—C(R¹⁰)—C(O)—X—Z, or R³ and R⁴ together with the nitrogen they are attached to form a substituted nitrogen-containing heterocyclyl; R⁵ and R⁶ are independently hydrogen, —(CH₂)ₓ—C(R¹⁰)—C(O)—X—Z, or R⁵ and R⁶ together with the nitrogen they are attached to form a substituted nitrogen-containing heterocyclyl; R¹⁰ is hydrogen, alkyl, aryl, or alkaryl; R¹¹, R¹², and R¹³ are independently C₁ to C₁ alkyl or alkaryl; R²⁰ and R²¹ are independently hydrogen, —(CH₂)ₓ—NR²⁰R²¹, —(CH₂)ₓ—C(R¹⁰)—C(O)—X—Z, or together with the nitrogen they are attached to form a substituted nitrogen-containing heterocyclyl; X is NH or O; Z is hydrogen, L¹-NR¹¹R¹²R¹³, L²-PR¹¹R¹²R¹³, L³-COOH, L⁴-SO₃H, L⁵-PO₃H, or a salt thereof; L¹, L², L³, L⁴, and L⁵ are independently C₁-C₁ alkylene or alkenylene; n is an integer of 1 to 10; and x is an integer of 1 to 10; wherein at least one of R³ and R⁴, or R⁵ and R⁶ together form a substituted nitrogen-containing heterocyclyl; and wherein at least one R² is —(CH₂)ₓ—C(R¹⁰)—C(O)—X—Z or —(CH₂)ₓ—NR²⁰R²¹ wherein one of R²⁰ or R²¹ is —(CH₂)ₓ—C(R¹⁰)—C(O)—X—Z.

The disclosure is further directed to a method of treating an aqueous medium, the method comprising contacting the aqueous medium with a multi-ionic compound disclosed herein.

The disclosure is also directed to a fabric softening composition comprising a multi-ionic compound disclosed herein.

Another aspect of the invention is a method of softening fabrics comprising contacting the fabric with an effective amount of a multi-ionic compound disclosed herein.

A further aspect of the invention is a method of inhibiting corrosion, biofilm growth, or bacterial growth in a system comprising contacting the fluid in the system with an effective amount of a multi-ionic compound disclosed herein.

Yet another aspect of the invention is a method of affecting the rheology of a composition comprising contacting the composition with an effective amount of a multi-ionic compound disclosed herein.

Other objects and features will be in part apparent and in part pointed out hereinafter.

### DETAILED DESCRIPTION OF THE INVENTION

Disclosed herein are compounds and compositions, methods of using the compounds and compositions for softening fabrics, inhibiting corrosion, inhibiting biofilm growth, inhibiting bacterial growth, and affecting the rheology of a composition, and processes for their preparation. The compounds and compositions are multi-ionic surfactants that can be used in various industries.

### Compounds and Compositions

Disclosed herein are multi-ionic surfactant compounds, the multi-ionic surfactant compounds corresponding to the structure of Formula 1, or a salt thereof:

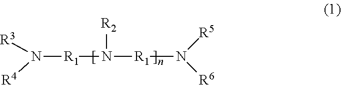

wherein R¹ is independently C₁-C₁₀ alkylene; R² is independently hydrogen, —(CH₂)ₓ—NR²⁰R²¹, or —(CH₂)ₓ—C(R¹⁰)—C(O)—X—Z; R³ and R⁴ are independently hydrogen, —(CH₂)ₓ—C(R¹⁰)—C(O)—X—Z, or R³ and R⁴ together with the nitrogen they are attached to form a substituted nitrogen-containing heterocyclyl; R⁵ and R⁶ are independently hydrogen, —(CH₂)ₓ—C(R¹⁰)—C(O)—X—Z, or R⁵ and R⁶ together with the nitrogen they are attached to form a substituted nitrogen-containing heterocyclyl; R¹⁰ is hydrogen, alkyl, aryl, or alkaryl; R¹, R¹², and R¹³ are independently C₁ to C₁₀ alkyl or alkaryl; R²⁰ and R²¹ are independently hydrogen, —(CH₂)ₓ—NR²⁰R²¹, —(CH₂)ₓ—C(R¹⁰)—C(O)—X—Z, or together with the nitrogen they are attached to form a substituted nitrogen-containing heterocyclyl; X is NH or O; Z is hydrogen, L¹-NR¹¹R¹²R¹³, L²-PR¹¹R¹²R¹³, L³-COOH, L⁴-SO₃H, L⁵-PO₃H, or a salt thereof; L¹, L², L³, L⁴, and L⁵ are independently C₁-C₁₀ alkylene or alkenylene; n is an integer of 1 to 10; and x is an integer of 1 to 10; wherein at least one of R³ and R⁴, or R⁵ and R⁶ together form a substituted nitrogen-containing heterocyclyl; and wherein at least one R² is —(CH₂)ₓ—C(R¹⁰)—C(O)—X—Z or —(CH₂)ₓ—NR²⁰R²¹ wherein one of R²⁰ or R²¹ is —(CH₂)ₓ—C(R¹⁰)—C(O)—X—Z;

For the compounds of Formula 1, preferably, R¹ can be independently C₂-C₆ alkylene, more preferably, R¹ can be independently C₂-C₄ alkylene, or most preferably, R¹ can be independently ethylene or propylene.

Additionally, compounds of Formula 1 can independently have one of R² be —(CH₂)ₓ—NR²⁰R²¹, or —(CH₂)ₓ—C(R¹⁰)—C(O)—X—Z.

When compounds of Formula 1 independently and preferably have one of R² be —(CH₂)ₓ—NR²⁰R²¹; then one of R²⁰ or R²¹ is —(CH₂)ₓ—C(R¹⁰)—C(O)—X—Z, wherein x is 1 or 2, R¹⁰ is hydrogen, X is NH, and Z is L¹-NR¹¹R¹²R¹³, wherein L¹ is C₁-C₄ alkylene, and R¹, R¹², and R¹³ are methyl.

When compounds of Formula 1 independently and more preferably have one of R² be —(CH₂)ₓ—C(R¹⁰)—C(O)—X—Z; then x is 1 or 2, R¹⁰ is hydrogen, X is NH, and Z is L¹-NR¹¹R¹²R¹³, wherein L¹ is C₁-C₄ alkylene, and R¹, R¹², and R¹³ are methyl.

In various compounds of Formula 1, x independently, can preferably be 1 to 6, more preferably, 1 to 4, or most preferably, 1 or 2.

Compounds of Formula 1 can have R³ and R⁴ together with the nitrogen they are attached to can form a substituted nitrogen-containing heterocyclyl, wherein the nitrogen-containing heterocyclyl is substituted with C₁₀ to C₂₄ alkyl or C₁₀ to C₂₄ alkenyl. Additionally, compounds of Formula 1 can have R³ and R⁴ together with the nitrogen they are attached to form:

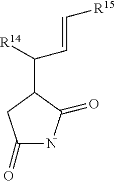

wherein R¹⁴ is a C₆ to C₁₂ alkyl and R¹⁵ is a C₆ to C₁₂ alkyl.

Also, the compounds of Formula 1 can have R⁵ and R⁶ together with the nitrogen they are attached to can form a substituted nitrogen-containing heterocyclyl, wherein the nitrogen-containing heterocyclyl is substituted with C₁₀ to C₂₄ alkyl or C₁₀ to C₂₄ alkenyl.

Further, the compounds of Formula 1 can have R¹⁰ be hydrogen, C₁ to C₃ alkyl or benzyl.

The compounds of Formula 1 can have X be 0 or NH.

Also, the compounds of Formula 1 can have Z be hydrogen, L³-COOH, L⁴-SO₃H, L⁵-PO₃H, or a salt thereof; or Z can be L¹-NR¹¹R¹²R¹³, L²-PR¹¹R¹²R¹³, L³-COOH, L⁴-SO₃H, L⁵-PO₃H, or a salt thereof.

The compounds of Formula 1 can preferably have L³, L⁴, and L⁵ be independently C₁ to C₆ alkylene; or more preferably, C₂ to C₃ alkylene.

Additionally, R¹¹, R¹², and R¹³ can be independently C₁ to C₁ alkyl or benzyl.

More particularly, the compounds of Formula 1 can preferably have Z be L¹-NR¹¹R¹²R¹³ when R¹¹, R¹², and R¹³ are independently C₁ to C₃ alkyl or benzyl; or more preferably Z can be L¹-NR¹¹R¹²R¹³ when R¹¹, R¹², and R¹³ are methyl.

In various compounds, n can preferably be an integer of 2 to 6, more preferably, 2 to 4, or most preferably, 4.

The multi-ionic surfactant compounds of Formula 1 can be a halogen salt (preferably, a chloride salt) or an acetate salt.

For example, multi-ionic surfactant compounds of Formula 1 can have the following structures:

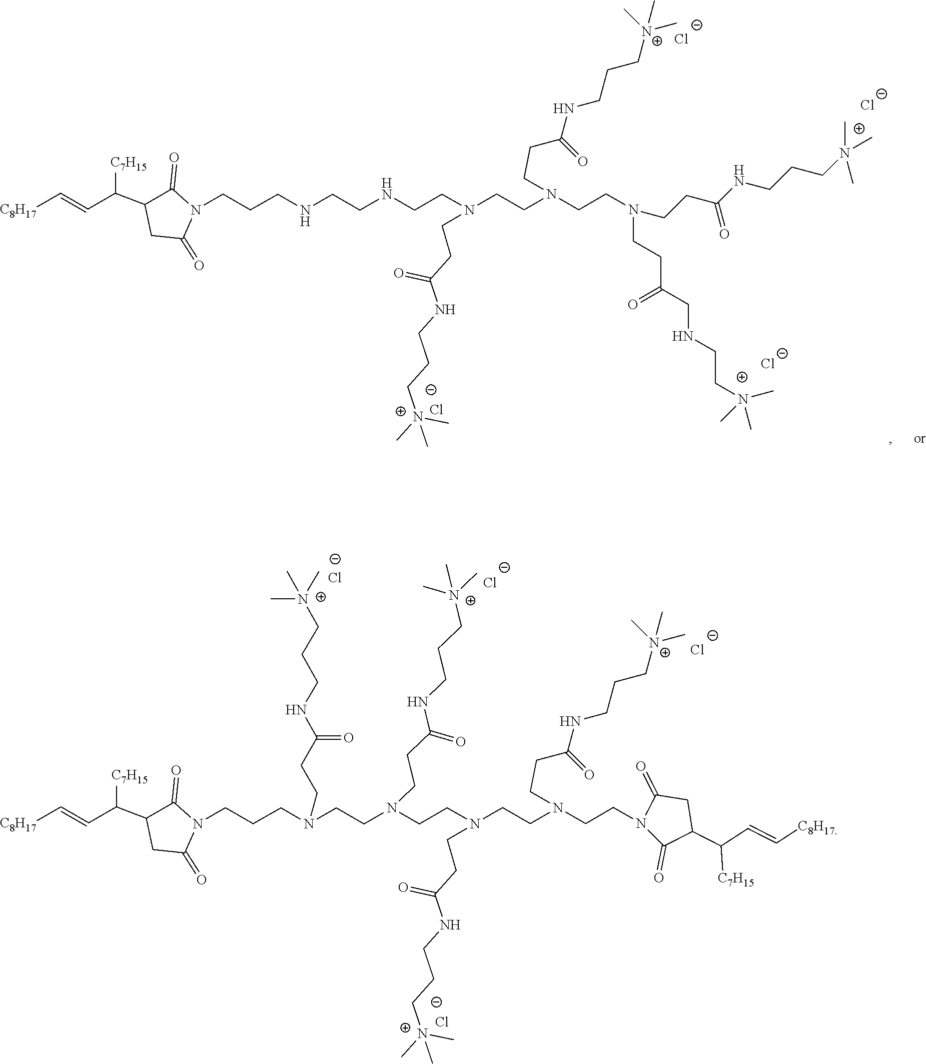

### Synthesis

The invention more particularly relates to the compounds that are derived by an aza-Michael addition reaction of an ASA (alkenyl succinic anhydride)-polyamine intermediate (obtained from imidation of polyamine and alkenyl succinic anhydride) and a cationic/anionic monomer.

An ASA-polyamine intermediate (III) is first obtained by an imidation reaction between a diamine or polyamine (I) and alkenyl succinic anhydride (II) as depicted in Scheme 1.

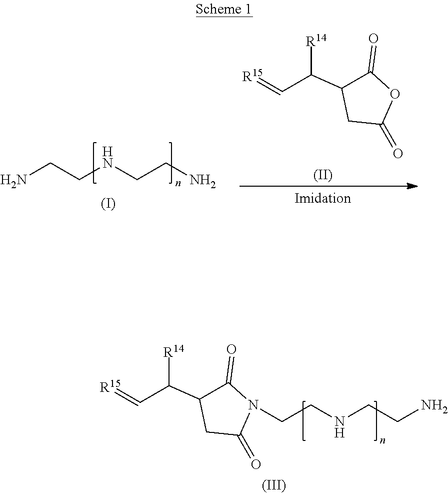

Wherein, n=1 to 100.

The second step involves an aza-Michael addition reaction between intermediate (III) and an α,β-unsaturated carbonyl compound containing at least one polar (charged) group (NR¹¹R¹²R¹³(+) X(−), —COOH, —SO₃H, —PO₃H, or a salt thereof) (IV) to afford multi-ionic compounds (V) as shown in Scheme 2.

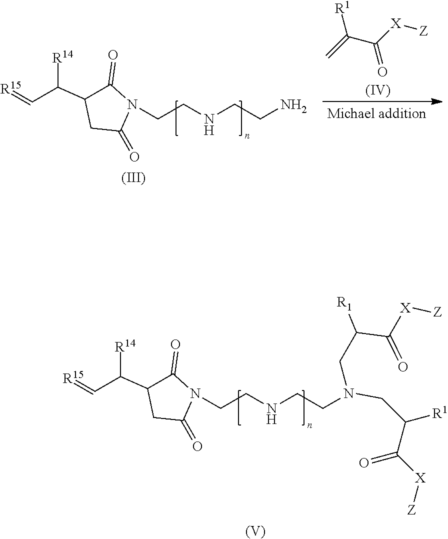

The generic synthesis reaction scheme for preparation of disclosed compounds using branched polyethyleneimine is shown in Scheme 3. The structure VI depicted below are a depiction of generalized reaction products.

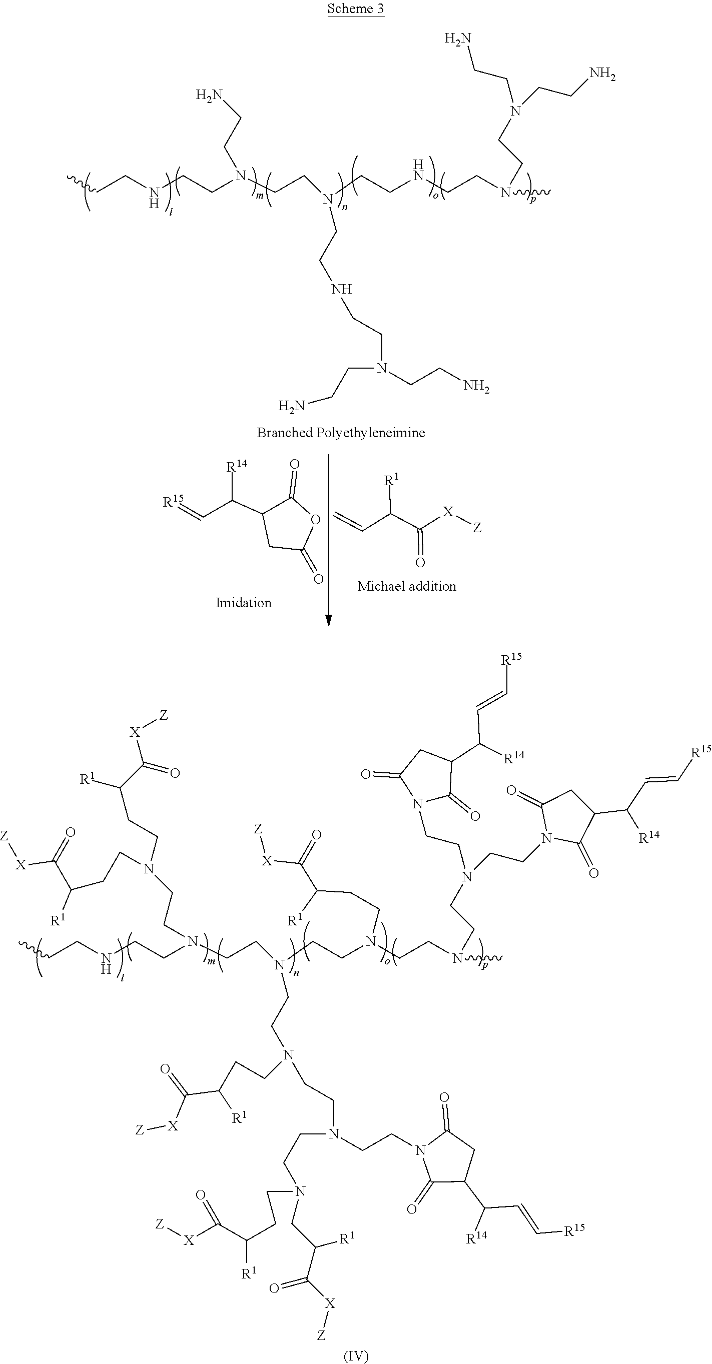

wherein:
k, l, m, m, o, p are independently an integer of 1-100,
X is independently NH or O,
R¹ is independently H, CH₃, or an unsubstituted, linear or branched C₂-C₁ alkyl group,
R¹⁴ and R¹⁵ are independently C₆ to C₁₂ alkyl;
Z is independently hydrogen, L¹-NR¹¹R¹²R¹³, L²-PR¹¹R¹²R¹³, L³-COOH, L⁴-SO₃H, L⁵-PO₃H, or a salt thereof;
L¹, L², L³, L⁴, and L⁵ are independently C₁-C₁₀ alkylene or alkenylene;
R¹¹, R¹², and R¹³ are independently C₁-C₁ alkyl group or benzyl group.

An α,β-unsaturated carbonyl compound containing at least one ionic group can be used as a Michael Acceptor. Used in the Examples herein are (3-Acrylamidopropyl)trimethylammonium chloride (APTAC) and 2-Acrylamido-2-methyl-1-propanesulfonic acid sodium salt (NaAMPS). Cationic monomers that could be used include [3-(Methacryloylamino)propyl]trimethylammonium chloride (MAPTAC), 2-(acryloyloxy)-N,N,N-trimethylethanaminium chloride (DMAEA-MCQ), N,N-dimethylaminoethyl acrylate benzyl chloride quaternary salt (DMAEA-BCQ), 2-(methacryloyloxy)-N,N,N-trimethylethan-1-aminium methyl sulfate (DMAEA-MSQ), 2-(acryloyloxy)-N,N,N-trimethylethanaminium chloride (DMAEA-MSQ), and the like. Anionic monomers that could be used include acrylic acid, methacrylic acid, itaconic acid, maleic acid, 3-(allyloxy)-2-hydroxypropane-1-sulfonate, and the like.

A polyalkyleneimine can be used as Michael Donor. Polyalkyleneimines can include, but are not limited to, branched, linear, or dendrimer polyethyleneimines. A few examples are diethylenetriamine, triethylenetetraamine, tetraethylenepentamine, pentaethylenehexamine, hexaethyleneheptamine, lupasol polyethyleneimines (different MW), tris(2-aminoethyl)amine, ethyleneamine E-100, and mixture thereof.

An alkenyl succinic anhydride (C8-alkyl+) can be used. A few examples are C12 ASA, C14 ASA, C16 ASA, C18 ASA, and C20-24 ASA.

### Methods

Another aspect of the invention is a method of softening fabrics comprising contacting the fabric with an effective amount of a multi-ionic compound of Formula 1 or the fabric softening composition disclosed herein.

A further aspect of the invention is a method of inhibiting corrosion, biofilm growth, or bacterial growth in a system comprising contacting the fluid in the system with an effective amount of a multi-ionic compound disclosed herein.

Yet another aspect of the invention is a method of affecting the rheology of a composition comprising contacting the composition with an effective amount of a multi-ionic compound disclosed herein.

Also disclosed are methods for inhibiting corrosion of a surface. The method comprises either: contacting the surface with an effective amount of a compound of formula (1) to inhibit corrosion on the surface; contacting the surface with a composition comprising an effective amount of the compound of formula (1) and a component comprising an organic solvent, a corrosion inhibitor, an asphaltene inhibitor, a paraffin inhibitor, a scale inhibitor, an emulsifier, a water clarifier, a dispersant, an emulsion breaker, a gas hydrate inhibitor, a biocide, a pH modifier, a surfactant, or a combination thereof to inhibit corrosion on the surface; or adding the compound or the composition to a fluid which contacts the surface to inhibit corrosion on the surface. The composition can be any composition as described herein.

The multi-ionic surfactant compound can be present in the fluid in an amount from about 1 ppm to about 5000 ppm; from about 1 ppm to about 4000 ppm; from about 1 ppm to about 3000 ppm; from about 1 ppm to about 2000 ppm; from about 1 ppm to about 1000 ppm; from about 1 ppm to about 800 ppm; from about 1 ppm to about 600 ppm; from about 1 ppm to about 500 ppm; from about 1 ppm to about 400 ppm; from about 1 ppm to about 300 ppm; from about 1 ppm to about 200 ppm; from about 1 ppm to about 100 ppm; from about 1 ppm to about 50 ppm; from about 5 ppm to about 5000 ppm; from about 5 ppm to about 4000 ppm; from about 5 ppm to about 3000 ppm; from about 5 ppm to about 2000 ppm; from about 5 ppm to about 1000 ppm; from about 5 ppm to about 800 ppm; from about 5 ppm to about 600 ppm; from about 5 ppm to about 500 ppm; from about 5 ppm to about 400 ppm; from about 5 ppm to about 300 ppm; from about 5 ppm to about 200 ppm; from about 5 ppm to about 100 ppm; from about 5 ppm to about 50 ppm; from about 10 ppm to about 5000 ppm; from about 10 ppm to about 4000 ppm; from about 10 ppm to about 3000 ppm; from about 10 ppm to about 2000 ppm; from about 10 ppm to about 1000 ppm; from about 10 ppm to about 800 ppm; from about 10 ppm to about 600 ppm; from about 10 ppm to about 500 ppm; from about 10 ppm to about 400 ppm; from about 10 ppm to about 300 ppm; preferably, from about 10 ppm to about 200 ppm; more preferably, from about 10 ppm to about 100 ppm; most preferably, from about 10 ppm to about 50 ppm, based on the total weight of the fluid.

The methods disclosed herein can have the surface be part of equipment used in an industrial system.

The industrial system can be a water recirculating system, a cooling water system, a boiler water system, a pulp slurry, a papermaking process, a ceramic slurry, a mixed solid/liquid system, or an oil-field system.

For the methods described herein, the surface can be part of equipment used in the production, transportation, storage, and/or separation of crude oil or natural gas.

In the methods disclosed, the equipment can comprise a pipeline, a storage vessel, a downhole injection tubing, a flow line, or an injection line.

For the methods described herein, the fluid can be used in the operation of the industrial system.

In the methods, the fluid can comprise seawater, produced water, fresh water, brackish water, drilling fluid, completion fluid, or a combination thereof.

The methods described herein can have the surface be part of equipment used in an industrial system. Preferably, the industrial system is a water recirculating system, a cooling water system, a boiler water system, a pulp slurry, a papermaking process, a ceramic slurry, a mixed solid/liquid system, or an oil-field system.

The methods can have the surface be part of equipment used in the production, transportation, storage, and/or separation of crude oil or natural gas. Preferably, the equipment comprises a pipeline, a storage vessel, downhole injection tubing, a flow line, or an injection line.

The methods described herein can have the fluid be used in the operation of the industrial system.

The fluid can comprise seawater, produced water, fresh water, brackish water, drilling fluid, completion fluid, or a combination thereof.

The disclosure is further directed to a method of treating an aqueous medium, the method comprising contacting the aqueous medium with a multi-ionic compound disclosed herein.

The disclosure is further directed to compositions comprising one or more of the multi-ionic surfactant compounds corresponding to the structure of Formula 1, or a salt thereof, as described herein.

The compositions can comprise from about 0.1 to about 20 wt. % of the multi-ionic surfactant compound based on the total weight of the composition.

The compositions described herein can comprise from about 0.1 to about 20 wt. % of one or more compounds of formula 1 in a solvent system.

In addition to the component, the compositions can also comprise water.

The composition comprises an effective amount of the compound of formula 1 and a component comprising an organic solvent, a corrosion inhibitor, an asphaltene inhibitor, a paraffin inhibitor, a scale inhibitor, an emulsifier, a water clarifier, a dispersant, an emulsion breaker, a gas hydrate inhibitor, a biocide, a pH modifier, a surfactant, or a combination thereof.

The compositions can comprise, for example, from about 0.1 to about 20 wt. % of one or more compounds of formula 1 and from about 80 to about 99.9 wt. % of the component; preferably, from about 0.1 to about 20 wt. % of one or more compounds of formula 1, from about 1 to about 60 wt. % of the component and from about 20 to about 98.9 wt. % water; more preferably, from about 10 to about 20 wt. % of one or more compounds of formula 1, from about 30 to about 40 wt. % of the component and from about 40 to about 60 wt. % water; or most preferably, from about 15 to about 20 wt. % of one or more compounds of formula 1, from about 1 to about 10 wt. % of the component and from about 70 to about 84 wt. % water.

The compositions can also comprise an organic solvent. The composition can comprise from about 1 to 80 wt. %, preferably, from about 5 to 50 wt. %, or most preferably, from about 10 to 35 wt. % of the one or more organic solvents, based on total weight of the composition. The organic solvent can comprise an alcohol, a hydrocarbon, a ketone, an ether, an alkylene glycol, a glycol ether, an amide, a nitrile, a sulfoxide, an ester, or a combination thereof. Examples of suitable organic solvents include, but are not limited to, methanol, ethanol, propanol, isopropanol, butanol, 2-ethylhexanol, hexanol, octanol, decanol, 2-butoxyethanol, methylene glycol, ethylene glycol, 1,2-propylene glycol, 1,3-propylene glycol, diethyleneglycol monomethyl ether, diethylene glycol monoethyl ether, ethylene glycol monobutyl ether, ethylene glycol dibutyl ether, pentane, hexane, cyclohexane, methylcyclohexane, heptane, decane, dodecane, diesel, toluene, xylene, heavy aromatic naphtha, cyclohexanone, diisobutylketone, diethyl ether, propylene carbonate, N-methylpyrrolidinone, N,N-dimethylformamide, or a combination thereof.

The compositions can further include a dispersant. The composition can comprise from about 0.1 to 10 wt. %, preferably, from about 0.5 to 5 wt. %, or most preferably, from about 0.5 to 4 wt. % of a dispersant, based on total weight of the composition. Suitable dispersants include, but are not limited to, aliphatic phosphonic acids with 2-50 carbons, such as hydroxyethyl diphosphonic acid, and aminoalkyl phosphonic acids, e.g. polyaminomethylene phosphonates with 2-10 N atoms e.g. each bearing at least one methylene phosphonic acid group; examples of the latter are ethylenediamine tetra(methylene phosphonate), diethylenetriamine penta(methylene phosphonate), and the triamine- and tetramine-polymethylene phosphonates with 2-4 methylene groups between each N atom, at least 2 of the numbers of methylene groups in each phosphonate being different. Other suitable dispersion agents include lignin, or derivatives of lignin such as lignosulfonate and naphthalene sulfonic acid and derivatives.

The compositions can also include an emulsion breaker. The composition can comprise from about 0.1 to 10 wt. %, preferably, from about 0.5 to 5 wt. %, or most preferably, from about 0.5 to 4 wt. % of an emulsion breaker, based on total weight of the composition. Suitable emulsion breakers include, but are not limited to, dodecylbenzylsulfonic acid (DDBSA), the sodium salt of xylenesulfonic acid (NAXSA), epoxylated and propoxylated compounds, anionic, cationic and nonionic surfactants, and resins, such as phenolic and epoxide resins.

The compositions can further include a demulsifier. Preferably, the demulsifier comprises an oxyalkylate polymer, such as a polyalkylene glycol. The demulsifier can constitute from about 0.1 to 10 wt. %, preferably, from about 0.5 to 5 wt. %, or most preferably, from about 0.5 to 4 wt. % of the composition, based on total weight of the composition. The demulsifier can constitute 0.5, 1, 1.5, 2, 2.5, 3, 3.5, 4, 4.5 or 5 wt. % of the composition.

The compositions can further include an emulsifier. The composition can comprise from about 0.1 to 10 wt. %, preferably, from about 0.5 to 5 wt. %, or most preferably, from about 0.5 to 4 wt. % of an emulsifier, based on total weight of the composition. Suitable emulsifiers include, but are not limited to, salts of carboxylic acids, products of acylation reactions between carboxylic acids or carboxylic anhydrides and amines, and alkyl, acyl and amide derivatives of saccharides (alkyl-saccharide emulsifiers).

The compositions can also include a biocide. The composition can comprise from about 0.1 to 10 wt. %, preferably, from about 0.5 to 5 wt. %, or most preferably, from about 0.5 to 4 wt. % of a biocide, based on total weight of the composition. Suitable biocides include, but are not limited to, oxidizing and non-oxidizing biocides. Suitable non-oxidizing biocides include, for example, aldehydes (e.g., formaldehyde, glutaraldehyde, and acrolein), amine-type compounds (e.g., quaternary amine compounds and cocodiamine), halogenated compounds (e.g., 2-bromo-2-nitropropane-3-diol (Bronopol) and 2-2-dibromo-3-nitrilopropionamide (DBNPA)), sulfur compounds (e.g., isothiazolone, carbamates, and metronidazole), and quaternary phosphonium salts (e.g., tetrakis(hydroxymethyl)-phosphonium sulfate (THPS)). Suitable oxidizing biocides include, for example, sodium hypochlorite, trichloroisocyanuric acids, dichloroisocyanuric acid, calcium hypochlorite, lithium hypochlorite, chlorinated hydantoins, stabilized sodium hypobromite, activated sodium bromide, brominated hydantoins, chlorine dioxide, ozone, and peroxides.

The compositions can also include a pH modifier. The composition can comprise from about 0.1 to 20 wt. %, preferably, from about 0.5 to 10 wt. %, or most preferably, from about 0.5 to 5 wt. % of a pH modifier, based on total weight of the composition. Suitable pH modifiers include, but are not limited to, alkali hydroxides, alkali carbonates, alkali bicarbonates, alkaline earth metal hydroxides, alkaline earth metal carbonates, alkaline earth metal bicarbonates and mixtures or combinations thereof. Exemplary pH modifiers include sodium hydroxide, potassium hydroxide, calcium hydroxide, calcium oxide, sodium carbonate, potassium carbonate, sodium bicarbonate, potassium bicarbonate, magnesium oxide, and magnesium hydroxide.

The compositions can include another surfactant. The composition can comprise from about 0.1 to 10 wt. %, preferably, from about 0.5 to 5 wt. %, or most preferably, from about 0.5 to 4 wt. % of a surfactant, based on total weight of the composition. Suitable surfactants include, but are not limited to, anionic surfactants and nonionic surfactants. Anionic surfactants include alkyl aryl sulfonates, olefin sulfonates, paraffin sulfonates, alcohol sulfates, alcohol ether sulfates, alkyl carboxylates and alkyl ether carboxylates, and alkyl and ethoxylated alkyl phosphate esters, and mono and dialkyl sulfosuccinates and sulfosuccinamates. Nonionic surfactants include alcohol alkoxylates, alkylphenol alkoxylates, block copolymers of ethylene, propylene and butylene oxides, alkyl dimethyl amine oxides, alkyl-bis(2-hydroxyethyl) amine oxides, alkyl amidopropyl dimethyl amine oxides, alkylamidopropyl-bis(2-hydroxyethyl) amine oxides, alkyl polyglucosides, polyalkoxylated glycerides, sorbitan esters and polyalkoxylated sorbitan esters, and alkoyl polyethylene glycol esters and diesters. Also included are betaines and sultanes, amphoteric surfactants such as alkyl amphoacetates and amphodiacetates, alkyl amphopropionates and amphodipropionates, and alkyliminodipropionate.

The compositions disclosed herein can further include additional functional agents or additives that provide a beneficial property. For example, additional agents or additives can be sequestrants, solubilizers, lubricants, buffers, cleaning agents, rinse aids, preservatives, binders, thickeners or other viscosity modifiers, processing aids, carriers, water-conditioning agents, foam inhibitors or foam generators, threshold agents or systems, aesthetic enhancing agents (i.e., dyes, odorants, perfumes), or other additives suitable for formulation with a composition, and mixtures thereof. Additional agents or additives will vary according to the particular composition being manufactured and its intend use as one skilled in the art will appreciate.

Alternatively, the compositions can not contain any of the additional agents or additives.

Further disclosed is a fabric softening composition comprising one or more of the multi-ionic surfactant compounds of Formula 1 disclosed herein.

The fabric softening compositions can further comprise one or more additional fabric softeners or cosofteners, silicones, solvents, emulsifiers, dispersants, emulsion breakers, demulsifiers, biocides, pH modifiers, or surfactants.

Also, the fabric softening composition can comprise from about 0.1 to about 50 wt. % of the multi-ionic surfactant compound based on the total weight of the composition.

The one or more additional fabric softeners of the fabric softening compositions can comprise diethyl ester dimethyl ammonium chloride, quaternized triethanolamine ditallow fatty acid esters, 1,2-dioleoyl-3-trimethylammonium propane, distearyldimethylammonium chloride, ethoxylated phosphate esters, or polydimethylsiloxane.

Additionally, the disclosure is directed to a fabric antistatic composition, a fabric conditioner composition, or a relaxant composition comprising one or more of the multi-ionic surfactant compounds of Formula 1.

The fabric antistatic, fabric conditioner, or relaxant compositions can comprise from about 0.1 to about 50 wt. % of the multi-ionic surfactant compound of Formula 1 based on the total weight of the composition.

Also disclosed herein are various compositions effective as surfactants, fabric softening agents, corrosion inhibitors, biofilm inhibitors, biocides, or rheology modifiers. The compositions comprise one or more of the multi-ionic surfactant compounds described herein.

The compositions can further comprise one or more additional corrosion inhibitors, an organic solvent, an asphaltene inhibitor, a paraffin inhibitor, a scale inhibitor, an emulsifier, a water clarifier, a dispersant, an emulsion breaker, a gas hydrate inhibitor, a biocide, a pH modifier, a surfactant, or a combination thereof.

The compositions can be formulated for inhibiting corrosion at a surface. The composition can comprise an effective amount of the compound of formula 1 and a component comprising an organic solvent, a corrosion inhibitor, an asphaltene inhibitor, a paraffin inhibitor, a scale inhibitor, an emulsifier, a water clarifier, a dispersant, an emulsion breaker, a gas hydrate inhibitor, a biocide, a pH modifier, a surfactant, or a combination thereof.

A compound used to enhance the corrosion performance of the composition can also be included in the anticorrosion composition. For example, thioglycolic acid, 3,3′-dithiopropioinic acid, thiosulfate, thiourea, 2-mercaptoethanol, L-cysteine, tert-butyl mercaptan, or a combination thereof can be included in the anticorrosion composition.

The compositions can further comprise a corrosion inhibitor in addition to the one or more compounds of formula 1. The composition can comprise from about 0.1 to 20 wt. %, preferably, 0.1 to 10 wt. %, or most preferably, 0.1 to 5 wt. % of the one or more additional corrosion inhibitors, based on total weight of the composition. A composition of the invention can comprise from 0 to 10 percent by weight of the one or more additional corrosion inhibitors, based on total weight of the composition. The composition can comprise 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10.0 wt. %, 10.5 wt. %, 11.0 wt. %, 11.5 wt. %, 12.0 wt. %, 12.5 wt. %, 13.0 wt. %, 13.5 wt. %, 14.0 wt. %, 14.5 wt. %, or 15.0 wt. % by weight of the one or more additional corrosion inhibitors, based on total weight of the composition. Each system can have its own requirements, and the weight percent of one or more additional corrosion inhibitors in the composition can vary with the system in which it is used.

The one or more additional corrosion inhibitors can comprise an imidazoline compound, a quaternary ammonium compound, a pyridinium compound, or a combination thereof.

The one or more additional corrosion inhibitors can comprise an imidazoline. The imidazoline can be, for example, imidazoline derived from a diamine, such as ethylene diamine (EDA), diethylene triamine (DETA), triethylene tetraamine (TETA) etc. and a long chain fatty acid such as tall oil fatty acid (TOFA). The imidazoline can be an imidazoline of Formula (I) or an imidazoline derivative. Representative imidazoline derivatives include an imidazolinium compound of Formula (II) or a bis-quaternized compound of Formula (III).

The one or more additional corrosion inhibitors can include an imidazoline of the following formula:

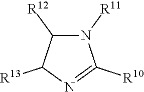

wherein R¹⁰ is a C₁-C₂₀ alkyl or a C₁-C₂₀ alkoxyalkyl group; R¹¹ is hydrogen, C₁-C₆ alkyl, C₁-C₆ hydroxyalkyl, or C₁-C₆ arylalkyl; and R¹² and R¹³ are independently hydrogen or a C₁-C₆ alkyl group. Preferably, the imidazoline includes an R¹⁰ which is the alkyl mixture typical in tall oil fatty acid (TOFA), and R¹¹, R¹² and R¹³ are each hydrogen.

The one or more additional corrosion inhibitors can include an imidazolinium compound of the following formulas:

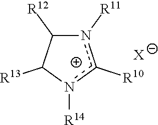

wherein R¹⁰ is a C₁-C₂₀ alkyl or a C₁-C₂₀ alkoxyalkyl group; R¹¹ and R¹⁴ are independently hydrogen, C₁-C₆ alkyl, C₁-C₆ hydroxyalkyl, or C₁-C₆ arylalkyl; R¹² and R¹³ are independently hydrogen or a C₁-C₆ alkyl group; and X⁻ is a halide (such as chloride, bromide, or iodide), carbonate, sulfonate, phosphate, or the anion of an organic carboxylic acid (such as acetate). Preferably, the imidazolinium compound includes 1-benzyl-1-(2-hydroxyethyl)-2-tall-oil-2-imidazolinium chloride.

The one or more additional corrosion inhibitors can comprise a bis-quaternized compound having the following formula:

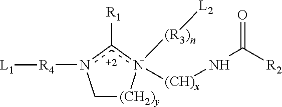

wherein:
R₁ and R₂ are each independently unsubstituted branched, chain or ring alkyl or alkenyl having from 1 to about 29 carbon atoms; partially or fully oxygenized, sulfurized, and/or phosphorylized branched, chain, or ring alkyl or alkenyl having from 1 to about 29 carbon atoms; or a combination thereof;
R₃ and R₄ are each independently unsubstituted branched, chain or ring alkylene or alkenylene having from 1 to about 29 carbon atoms; partially or fully oxygenized, sulfurized, and/or phosphorylized branched, chain, or ring alkylene or alkenylene having from 1 to about 29 carbon atoms; or a combination thereof;
L₁ and L₂ are each independently absent, H, —COOH, —SO₃H, —PO₃H₂, —COOR₅, —CONH₂, —CONHR₅, or —CON(R₅)₂;
R₅ is each independently a branched or unbranched alkyl, aryl, alkylaryl, alkylheteroaryl, cycloalkyl, or heteroaryl group having from 1 to about 10 carbon atoms;
n is 0 or 1, and when n is 0, L₂ is absent or H;
x is from 1 to about 10; and
y is from 1 to about 5. Preferably, R₁ and R₂ are each independently C₆-C₂₂ alkyl, C₈-C₂₀ alkyl, C₁₂-C₁₈ alkyl, C₁₆-C₁₈ alkyl, or a combination thereof; R₃ and R₄ are C₁-C₁₀ alkylene, C₂-C₈ alkylene, C₂-C₆ alkylene, or C₂-C₃ alkylene; n is 0 or 1; x is 2; y is 1; R₃ and R₄ are —C₂H₂—; L¹ is —COOH, —SO₃H, or —PO₃H₂; and L₂ is absent, H, —COOH, —SO₃H, or —PO₃H₂. For example, R₁ and R₂ can be derived from a mixture of tall oil fatty acids and are predominantly a mixture of C₁₇H₃₃ and C₁₇H₃₁ or can be C₁₆-C₁₈ alkyl; R₃ and R₄ can be C₂-C₃ alkylene such as —C₂H₂—; n is 1 and L₂ is —COOH or n is 0 and L₂ is absent or H; x is 2; y is 1; R₃ and R₄ are —C₂H₂—; and L¹ is —COOH.

It should be appreciated that the number of carbon atoms specified for each group of the formula above refers to the main chain of carbon atoms and does not include carbon atoms that may be contributed by substituents.

The one or more additional corrosion inhibitors can comprise a bis-quaternized imidazoline compound having the formula (III) wherein R₁ and R₂ are each independently C₆-C₂₂ alkyl, C₈-C₂₀ alkyl, C₁₂-C₁₈ alkyl, or C₁₆-C₁₈ alkyl or a combination thereof; R₄ is C₁-C₁₀ alkylene, C₂-C₈ alkylene, C₂-C₆ alkylene, or C₂-C₃ alkylene; x is 2; y is 1; n is 0; L¹ is —COOH, —SO₃H, or —PO₃H₂; and L₂ is absent or H. Preferably, a bis-quaternized compound has the formula (III) wherein R₁ and R₂ are each independently C₁₆-C₁₈ alkyl; R₄ is —C₂H₂—; x is 2; y is 1; n is 0; L¹ is —COOH, —SO₃H, or —PO₃H₂ and L₂ is absent or H.

The one or more additional corrosion inhibitors can be a quaternary ammonium compound of the following formula:

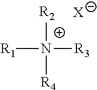

wherein R₁, R₂, and R₃ are independently C₁ to C₂₀ alkyl, R⁴ is methyl or benzyl, and X⁻ is a halide or methosulfate.

Suitable alkyl, hydroxyalkyl, alkylaryl, arylalkyl or aryl amine quaternary salts include those alkylaryl, arylalkyl and aryl amine quaternary salts of the formula [N+R^{5a}R^{6a}R^{7a}R^{8a}][X⁻] wherein R^{5a}, R^{6a}, R^{7a}, and R^{8a} contain one to 18 carbon atoms, and X is Cl, Br or I. For the quaternary salts, R^{5a}, R^{6a}, R^{7a}, and R^{8a} can each be independently alkyl (e.g., C₁-C₁₈ alkyl), hydroxyalkyl (e.g., C₁-C₁₈ hydroxyalkyl), and arylalkyl (e.g., benzyl). The mono or polycyclic aromatic amine salt with an alkyl or alkylaryl halide include salts of the formula [N+R^{5a}R^{6a}R^{7a}R^{8a}][X⁻] wherein R^{5a}, R^{6a}, R^{7a}, and R^{8a} contain one to 18 carbon atoms and at least one aryl group, and X is Cl, Br or I.

Suitable quaternary ammonium salts include, but are not limited to, a tetramethyl ammonium salt, a tetraethyl ammonium salt, a tetrapropyl ammonium salt, a tetrabutyl ammonium salt, a tetrahexyl ammonium salt, a tetraoctyl ammonium salt, a benzyltrimethyl ammonium salt, a benzyltriethyl ammonium salt, a phenyltrimethyl ammonium salt, a phenyltriethyl ammonium salt, a cetyl benzyldimethyl ammonium salt, a hexadecyl trimethyl ammonium salt, a dimethyl alkyl benzyl quaternary ammonium salt, a monomethyl dialkyl benzyl quaternary ammonium salt, or a trialkyl benzyl quaternary ammonium salt, wherein the alkyl group has about 6 to about 24 carbon atoms, about 10 and about 18 carbon atoms, or about 12 to about 16 carbon atoms. The quaternary ammonium salt can be a benzyl trialkyl quaternary ammonium salt, a benzyl triethanolamine quaternary ammonium salt, or a benzyl dimethylaminoethanolamine quaternary ammonium salt.

The one or more additional corrosion inhibitors can comprise a pyridinium salt such as those represented by the following formula:

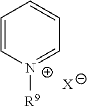

wherein R⁹ is an alkyl group, an aryl group, or an arylalkyl group, wherein said alkyl groups have from 1 to about 18 carbon atoms and X⁻ is a halide such as chloride, bromide, or iodide. Among these compounds are alkyl pyridinium salts and alkyl pyridinium benzyl quats. Exemplary compounds include methyl pyridinium chloride, ethyl pyridinium chloride, propyl pyridinium chloride, butyl pyridinium chloride, octyl pyridinium chloride, decyl pyridinium chloride, lauryl pyridinium chloride, cetyl pyridinium chloride, benzyl pyridinium chloride and an alkyl benzyl pyridinium chloride, preferably wherein the alkyl is a C₁-C₆ hydrocarbyl group. Preferably, the pyridinium compound includes benzyl pyridinium chloride.

The one or more additional corrosion inhibitors can include additional corrosion inhibitors such as phosphate esters, monomeric or oligomeric fatty acids, or alkoxylated amines.

The one or more additional corrosion inhibitors can comprise a phosphate ester. Suitable mono-, di- and tri-alkyl as well as alkylaryl phosphate esters and phosphate esters of mono, di, and triethanolamine typically contain between from 1 to about 18 carbon atoms. Preferred mono-, di- and trialkyl phosphate esters, alkylaryl or arylalkyl phosphate esters are those prepared by reacting a C₃-C₁₈ aliphatic alcohol with phosphorous pentoxide. The phosphate intermediate interchanges its ester groups with triethylphosphate producing a broader distribution of alkyl phosphate esters.

Alternatively, the phosphate ester can be made by admixing with an alkyl diester, a mixture of low molecular weight alkyl alcohols or diols. The low molecular weight alkyl alcohols or diols preferably include C₆ to C₁₀ alcohols or diols. Further, phosphate esters of polyols and their salts containing one or more 2-hydroxyethyl groups, and hydroxylamine phosphate esters obtained by reacting polyphosphoric acid or phosphorus pentoxide with hydroxylamines such as diethanolamine or triethanolamine are preferred.

The one or more additional corrosion inhibitors can include a monomeric or oligomeric fatty acid. Preferred monomeric or oligomeric fatty acids are C₁₄-C₂₂ saturated and unsaturated fatty acids as well as dimer, trimer and oligomer products obtained by polymerizing one or more of such fatty acids.

The one or more additional corrosion inhibitors can comprise an alkoxylated amine. The alkoxylated amine can be an ethoxylated alkyl amine. The alkoxylated amine can be ethoxylated tallow amine.

The compositions can further comprise an organic sulfur compound, such as a mercaptoalkyl alcohol, mercaptoacetic acid, thioglycolic acid, 3,3′-dithiodipropionic acid, sodium thiosulfate, thiourea, L-cysteine, tert-butyl mercaptan, sodium thiosulfate, ammonium thiosulfate, sodium thiocyanate, ammonium thiocyanate, sodium metabisulfite, or a combination thereof. Preferably, the mercaptoalkyl alcohol comprises 2-mercaptoethanol. The organic sulfur compound can constitute 0.5 to 15 wt. % of the composition, based on total weight of the composition, preferably about 1 to about 10 wt. % and more preferably about 1 to about 5 wt. %. The organic sulfur compound can constitute 1, 2, 3, 4, 5, 6, 7, 8, 9, 10, 11, 12, 13, 14 or 15 wt. % of the composition.

The compositions can be substantially free of or free of any organic sulfur compound. A composition is substantially free of any organic sulfur compound if it contains an amount of organic sulfur compound less than 0.50 wt. % preferably less than 0.10 wt. %, and more preferably less than 0.01 wt. %.

The compositions can also include an asphaltene inhibitor. The composition can comprise from about 0.1 to 10 wt. %, from about 0.1 to 5 wt. %, or from about 0.5 to 4 wt. % of an asphaltene inhibitor, based on total weight of the composition. Suitable asphaltene inhibitors include, but are not limited to, aliphatic sulfonic acids; alkyl aryl sulfonic acids; aryl sulfonates; lignosulfonates; alkylphenol/aldehyde resins and similar sulfonated resins; polyolefin esters; polyolefin imides; polyolefin esters with alkyl, alkylenephenyl or alkylenepyridyl functional groups; polyolefin amides; polyolefin amides with alkyl, alkylenephenyl or alkylenepyridyl functional groups; polyolefin imides with alkyl, alkylenephenyl or alkylenepyridyl functional groups; alkenyl/vinyl pyrrolidone copolymers; graft polymers of polyolefins with maleic anhydride or vinyl imidazole; hyperbranched polyester amides; polyalkoxylated asphaltenes, amphoteric fatty acids, salts of alkyl succinates, sorbitan monooleate, and polyisobutylene succinic anhydride.

The compositions can include a paraffin inhibitor. The composition can comprise from about 0.1 to 10 wt. %, from about 0.1 to 5 wt. %, or from about 0.5 to 4 wt. % of a paraffin inhibitor, based on total weight of the composition. Suitable paraffin inhibitors include, but are not limited to, paraffin crystal modifiers, and dispersant/crystal modifier combinations. Suitable paraffin crystal modifiers include, but are not limited to, alkyl acrylate copolymers, alkyl acrylate vinylpyridine copolymers, ethylene vinyl acetate copolymers, maleic anhydride ester copolymers, branched polyethylenes, naphthalene, anthracene, microcrystalline wax and/or asphaltenes. Suitable paraffin dispersants include, but are not limited to, dodecyl benzene sulfonate, oxyalkylated alkylphenols, and oxyalkylated alkylphenolic resins.

The compositions can also include a scale inhibitor. The composition can comprise from about 0.1 to 20 wt. %, from about 0.5 to 10 wt. %, or from about 1 to 10 wt. % of a scale inhibitor, based on total weight of the composition. Suitable scale inhibitors include, but are not limited to, phosphates, phosphate esters, phosphoric acids, phosphonates, phosphonic acids, polyacrylamides, salts of acrylamidomethyl propane sulfonate/acrylic acid copolymer (AMPS/AA), phosphinated maleic copolymer (PHOS/MA), and salts of a polymaleic acid/acrylic acid/acrylamidomethyl propane sulfonate terpolymer (PMA/AA/AMPS).

The compositions can also include a water clarifier. The composition can comprise from about 0.1 to 10 wt. %, from about 0.5 to 5 wt. %, or from about 0.5 to 4 wt. % of a water clarifier, based on total weight of the composition. Suitable water clarifiers include, but are not limited to, inorganic metal salts such as alum, aluminum chloride, and aluminum chlorohydrate, or organic polymers such as acrylic acid based polymers, acrylamide based polymers, polymerized amines, alkanolamines, thiocarbamates, and cationic polymers such as diallyldimethylammonium chloride (DADMAC).

The compositions can further include a hydrogen sulfide scavenger. The composition can comprise from about 1 to 50 wt. %, from about 1 to 40 wt. %, or from about 1 to 30 wt. % of a hydrogen sulfide scavenger, based on total weight of the composition. Suitable additional hydrogen sulfide scavengers include, but are not limited to, oxidants (e.g., inorganic peroxides such as sodium peroxide or chlorine dioxide); aldehydes (e.g., of 1-10 carbons such as formaldehyde, glyoxal, glutaraldehyde, acrolein, or methacrolein; triazines (e.g., monoethanolamine triazine, monomethylamine triazine, and triazines from multiple amines or mixtures thereof); condensation products of secondary or tertiary amines and aldehydes, and condensation products of alkyl alcohols and aldehydes.

The compositions can also include a gas hydrate inhibitor. The composition can comprise from about 0.1 to 25 wt. %, from about 0.5 to 20 wt. %, or from about 1 to 10 wt. % of a gas hydrate inhibitor, based on total weight of the composition. Suitable gas hydrate inhibitors include, but are not limited to, thermodynamic hydrate inhibitors (THI), kinetic hydrate inhibitors (KHI), and anti-agglomerates (AA). Suitable thermodynamic hydrate inhibitors include, but are not limited to, sodium chloride, potassium chloride, calcium chloride, magnesium chloride, sodium bromide, formate brines (e.g. potassium formate), polyols (such as glucose, sucrose, fructose, maltose, lactose, gluconate, monoethylene glycol, diethylene glycol, triethylene glycol, mono-propylene glycol, dipropylene glycol, tripropylene glycols, tetrapropylene glycol, monobutylene glycol, dibutylene glycol, tributylene glycol, glycerol, diglycerol, triglycerol, and sugar alcohols (e.g. sorbitol, mannitol)), methanol, propanol, ethanol, glycol ethers (such as diethyleneglycol monomethylether, ethyleneglycol monobutylether), and alkyl or cyclic esters of alcohols (such as ethyl lactate, butyl lactate, methylethyl benzoate).

The compositions can further include a kinetic hydrate inhibitor. The composition can comprise from about 0.1 to 25 wt. %, from about 0.5 to 20 wt. %, or from about 1 to 10 wt. % of a kinetic hydrate inhibitor, based on total weight of the composition. Suitable kinetic hydrate inhibitors and anti-agglomerates include, but are not limited to, polymers and copolymers, polysaccharides (such as hydroxyethylcellulose (HEC), carboxymethylcellulose (CMC), starch, starch derivatives, and xanthan), lactams (such as polyvinylcaprolactam, polyvinyl lactam), pyrrolidones (such as polyvinyl pyrrolidone of various molecular weights), surfactants (such as fatty acid salts, ethoxylated alcohols, propoxylated alcohols, sorbitan esters, ethoxylated sorbitan esters, polyglycerol esters of fatty acids, alkyl glucosides, alkyl polyglucosides, alkyl sulfates, alkyl sulfonates, alkyl ester sulfonates, alkyl aromatic sulfonates, alkyl betaine, alkyl amido betaines), hydrocarbon based dispersants (such as lignosulfonates, iminodisuccinates, polyaspartates), amino acids, and proteins.

Additionally, the compounds of Formula 1 can be formulated into compositions comprising the following components. These formulations include the ranges of the components listed and can optionally include additional agents.

TABLE 1

| Component | 1 | 2 | 3 | 4 | 5 | 6 | 7 | 8 | 9 | 10 | 11 | 12 |
|---|---|---|---|---|---|---|---|---|---|---|---|---|
| Compound of | 0.1-20 | 0.1-20 | 0.1-20 | 0.1-20 | 0.1-20 | 0.1-20 | 10-20 | 10-20 | 10-20 | 10-20 | 10-20 | 0.1-20 |
| Formula 1 | | | | | | | | | | | | |
| Organic solvent | 5-40 | — | 5-50 | — | 5-50 | 5-50 | 5-40 | — | 5-50 | — | — | 10-20 |
| Additional | 0.1-20 | 0.1-20 | — | — | — | — | 0.1-20 | 0.1-20 | — | — | — | 0.1-20 |
| corrosion | | | | | | | | | | | | |
| inhibitor | | | | | | | | | | | | |
| Asphaltene | 0.1-5 | 0.1-5 | 0.1-5 | 0.1-5 | — | — | 0.1-5 | 0.1-5 | 0.1-5 | — | — | 0.1-5 |
| inhibitor | | | | | | | | | | | | |
| Scale inhibitor | 1-10 | 1-10 | 1-10 | 1-10 | 1-10 | — | 1-10 | 1-10 | 1-10 | 1-10 | — | 1-10 |
| Gas hydrate | — | — | — | — | — | — | — | — | — | — | — | 0.1-25 |
| inhibitor | | | | | | | | | | | | |
| Biocide | 0.5-5 | 0.5-5 | 0.5-5 | 0.5-5 | 0.5-5 | 0.5-5 | 0.5-5 | 0.5-5 | 0.5-5 | 0.5-5 | 0.5-5 | |
| Water | 0.00 | 0-40 | 0-10 | 0-60 | 0-15 | 0-25 | 0.00 | 0-40 | 0-10 | 0-65 | 0-75 | |

TABLE 2

| Component | 13 | 14 | 15 | 16 | 17 | 18 | 19 | 20 | 21 | 22 | 23 | 24 |
|---|---|---|---|---|---|---|---|---|---|---|---|---|
| Compound of | 0.1-20 | 0.1-20 | 0.1-20 | 0.1-20 | 0.1-20 | 0.1-20 | 10-20 | 10-20 | 10-20 | 10-20 | 10-20 | 10-20 |
| Formula 1 | | | | | | | | | | | | |
| Organic solvent | — | 10-20 | — | 10-35 | 10-35 | — | 10-15 | — | — | 10-35 | 10-35 | — |
| Additional | 0.1-20 | 0.1-20 | 0.1-20 | 0.1-20 | 0.1-20 | 0.1-20 | 0.1-20 | 0.1-20 | 0.1-20 | 0.1-20 | 0.1-20 | 0.1-20 |
| corrosion | | | | | | | | | | | | |
| inhibitor | | | | | | | | | | | | |
| Asphaltene | 0.1-5 | — | — | — | — | — | 0.1-5 | — | — | — | — | — |
| inhibitor | | | | | | | | | | | | |
| Scale inhibitor | 1-10 | 1-10 | — | — | 1-10 | — | 1-10 | 1-10 | — | — | — | 1-10 |
| Gas hydrate | 0.1-25 | 0.1-25 | 0.1-25 | — | — | — | 0.1-25 | 0.1-25 | 0.1-25 | — | 0.1-25 | — |
| inhibitor | | | | | | | | | | | | |
| Biocide | — | — | — | — | — | 0.5-5 | 0.5-5 | 0.5-5 | 0.5-5 | 0.5-5 | — | — |
| Water | 0-20 | 0-5 | 0-35 | 0-25 | 0-15 | 0-55 | 0.00 | 0-20 | 0-30 | 0-20 | 0.00 | 0-50 |

The compounds/compositions can be used for inhibiting corrosion in oil and gas applications such as by treating a gas or liquid stream with an effective amount of a compound or composition as described herein. The compounds and compositions can be used in any industry where it is desirable to inhibit corrosion at a surface.

The compounds/compositions can be used in water systems, condensate/oil systems/gas systems, or any combination thereof. For example, the compounds/compositions can be used in controlling scale on heat exchanger surfaces.

The compounds/compositions can be applied to a gas or liquid produced, or used in the production, transportation, storage, and/or separation of crude oil or natural gas.

The compounds/compositions can be applied to a gas stream used or produced in a coal-fired process, such as a coal-fired power plant.

The compounds/compositions can be applied to a gas or liquid produced or used in a waste-water process, a farm, a slaughter house, a land-fill, a municipality waste-water plant, a coking coal process, or a biofuel process.

A fluid to which the compounds/compositions can be introduced can be an aqueous medium. The aqueous medium can comprise water, gas, and optionally liquid hydrocarbon.

A fluid to which the compounds/compositions can be introduced can be a liquid hydrocarbon. The liquid hydrocarbon can be any type of liquid hydrocarbon including, but not limited to, crude oil, heavy oil, processed residual oil, bituminous oil, coker oils, coker gas oils, fluid catalytic cracker feeds, gas oil, naphtha, fluid catalytic cracking slurry, diesel fuel, fuel oil, jet fuel, gasoline, and kerosene.

The fluid or gas can be a refined hydrocarbon product.

A fluid or gas treated with a compound/composition can be at any selected temperature, such as ambient temperature or an elevated temperature. The fluid (e.g., liquid hydrocarbon) or gas can be at a temperature of from about 40° C. to about 250° C. The fluid or gas can be at a temperature of from −50° C. to 300° C., 0° C. to 200° C., 10° C. to 100° C., or 20° C. to 90° C. The fluid or gas can be at a temperature of 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. The fluid or gas can be at a temperature of 85° C., 86° C., 87° C., 88° C., 89° C., 90° C., 91° C., 92° C., 93° C., 94° C., 95° C., 96° C., 97° C., 98° C., 99° C., or 100° C.

The compounds/compositions can be added to a fluid at various levels of water cut. For example, the water cut can be from 0% to 100% volume/volume (v/v), from 1% to 80% v/v, or from 1% to 60% v/v. The fluid can be an aqueous medium that contains various levels of salinity. The fluid can have a salinity of 0% to 25%, about 1% to 24%, or about 10% to 25% weight/weight (w/w) total dissolved solids (TDS).

The fluid or gas in which the compounds/compositions are introduced can be contained in and/or exposed to many different types of apparatuses. For example, the fluid or gas can be contained in an apparatus that transports fluid or gas from one point to another, such as an oil and/or gas pipeline. The apparatus can be part of an oil and/or gas refinery, such as a pipeline, a separation vessel, a dehydration unit, or a gas line. The fluid can be contained in and/or exposed to an apparatus used in oil extraction and/or production, such as a wellhead. The apparatus can be part of a coal-fired power plant. The apparatus can be a scrubber (e.g., a wet flue gas desulfurizer, a spray dry absorber, a dry sorbent injector, a spray tower, a contact or bubble tower, or the like). The apparatus can be a cargo vessel, a storage vessel, a holding tank, or a pipeline connecting the tanks, vessels, or processing units.

The compounds/compositions can be introduced into a fluid or gas by any appropriate method for ensuring dispersal through the fluid or gas.

The compounds/compositions can be added to the hydrocarbon fluid before the hydrocarbon fluid contacts the surface.

The compounds/compositions can be added at a point in a flow line upstream from the point at which corrosion prevention is desired.

The compounds/compositions can be injected using mechanical equipment such as chemical injection pumps, piping tees, injection fittings, atomizers, quills, and the like.

The compounds/compositions of the invention can be introduced with or without one or more additional polar or non-polar solvents depending upon the application and requirements.

The compounds/compositions can be pumped into an oil and/or gas pipeline using an umbilical line. A capillary injection system can be used to deliver the compounds/compositions to a selected fluid.

The compounds/compositions can be introduced into a liquid and mixed.

The compounds/compositions can be injected into a gas stream as an aqueous or non-aqueous solution, mixture, or slurry.

The fluid or gas can be passed through an absorption tower comprising compounds/compositions.

The compounds/compositions can be applied continuously, in batch, or a combination thereof. The compounds/compositions doses can be continuous to prevent corrosion. The compounds/compositions doses can be intermittent (i.e., batch treatment) or the compounds/compositions doses can be continuous/maintained and/or intermittent to inhibit corrosion.

The flow rate of a flow line in which the compound/composition is used can be between 0 and 100 feet per second, or between 0.1 and 50 feet per second. The compounds/compositions can also be formulated with water in order to facilitate addition to the flow line.

The compounds/compositions of the invention can be used for inhibiting corrosion in other applications.

The compounds/compositions are useful for corrosion inhibition of containers, processing facilities, or equipment in the food service or food processing industries. The compounds/compositions have particular value for use on food packaging materials and equipment, and especially for cold or hot aseptic packaging. Examples of process facilities in which the compounds/compositions can be employed include a milk line dairy, a continuous brewing system, food processing lines such as pumpable food systems and beverage lines, ware wash machines, low temperature ware wash machines, dishware, bottle washers, bottle chillers, warmers, third sink washers, processing equipment such as tanks, vats, lines, pumps and hoses (e.g., dairy processing equipment for processing milk, cheese, ice cream and other dairy products), and transportation vehicles. The compounds/compositions can be used to inhibit corrosion in tanks, lines, pumps, and other equipment used for the manufacture and storage of soft drink materials, and also used in the bottling or containers for the beverages.

The compounds/compositions can also be used on or in other industrial equipment and in other industrial process streams such as heaters, cooling towers, boilers, retort waters, rinse waters, aseptic packaging wash waters, and the like. The compounds/compositions can be used to treat surfaces in recreational waters such as in pools, spas, recreational flumes and water slides, fountains, and the like.

The compounds/compositions can be used to inhibit the corrosion of metal surfaces contacted with cleaners in surfaces found in janitorial and/or housekeeping applications, food processing equipment and/or plant applications, and in laundry applications. For example, the corrosion of washers, such as tunnel washers for washing textiles, can be inhibited according to methods disclosed herein.

The compounds/compositions can be used or applied in combination with low temperature dish and/or warewash sanitizing final rinse, toilet bowl cleaners, and laundry bleaches. The compounds, compositions and methods can be used to treat metal surfaces, such as ware, cleaned and/or sanitized with corrosive sources.

The compounds, compositions and methods disclosed herein protect surfaces from corrosion caused by hypochlorite bleach. A method can include providing the corrosion inhibitor compounds/compositions to a surface treated with a hypochlorite solution in order to inhibit corrosion caused by the hypochlorite solution. The method can include preparing an aqueous use composition of the present corrosion inhibitor composition. The method can further include contacting a surface, such as a hard metal surface, in need of corrosion inhibition due to contact with a hypochlorite solution.

The compounds/compositions can be dispensed by immersing either intermittently or continuously in water. The composition can then dissolve, for example, at a controlled or predetermined rate. The rate can be effective to maintain a concentration of dissolved agent that is effective for use according to the methods disclosed herein.

In the methods described herein, the multi-ionic surfactant compound can be present in the fluid in an amount from about 1 ppm to about 5000 ppm; preferably, from about 20 ppm to about 200 ppm.

For the methods described herein, the multi-ionic surfactant compound of Formula 1 can be present in an amount from about 0.1 ppm to about 10000 ppm, from about 0.1 ppm to about 5000 ppm, from about 0.1 ppm to about 3000 ppm, from about 0.1 ppm to about 2000 ppm, from about 0.1 ppm to about 1500 ppm, from about 0.1 ppm to about 1000 ppm, from about 0.1 ppm to about 500 ppm, from about 0.5 ppm to about 5000 ppm, from about 0.5 ppm to about 4000 ppm, from about 0.5 ppm to about 3000 ppm, from about 0.5 ppm to about 2500 ppm, from about 0.5 ppm to about 2000 ppm, from about 0.5 ppm to about 1500 ppm, from about 0.5 ppm to about 1000 ppm, from about 0.5 ppm to about 500 ppm, from about 1 ppm to about 5000 ppm, from about 1 ppm to about 4000 ppm, from about 1 ppm to about 3000 ppm, from about 1 ppm to about 2500 ppm, from about 1 ppm to about 2000 ppm, from about 1 ppm to about 1500 ppm, from about 1 ppm to about 1000 ppm, from about 1 ppm to about 500 ppm, from about 1 ppm to about 100 ppm, or from about 1 ppm to about 10 ppm, based on the total weight of the fluid in contact with the surface.

The term “alkyl,” as used herein, refers to a linear or branched hydrocarbon radical, preferably having 1 to 32 carbon atoms (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, or 32 carbons). Alkyl groups include, but are not limited to, methyl, ethyl, n-propyl, isopropyl, n-butyl, iso-butyl, secondary-butyl, and tertiary-butyl. Alkyl groups may be unsubstituted or substituted by one or more suitable substituents, as defined above.

The term “alkenyl,” as used herein, refers to a straight or branched hydrocarbon radical, preferably having 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, or 32 carbons, and having one or more carbon-carbon double bonds. Alkenyl groups include, but are not limited to, ethenyl, 1-propenyl, 2-propenyl (allyl), iso-propenyl, 2-methyl-1-propenyl, 1-butenyl, and 2-butenyl. Alkenyl groups may be unsubstituted or substituted by one or more suitable substituents, as defined above.

The term “alkoxy,” as used herein, refers to an alkyl group, as defined herein, appended to the parent molecular moiety through an oxygen atom.

The term “aryl,” as used herein, means monocyclic, bicyclic, or tricyclic aromatic radicals such as phenyl, naphthyl, tetrahydronaphthyl, indanyl and the like; optionally substituted by one or more suitable substituents, preferably 1 to 5 suitable substituents, as defined above.

The term “cycloalkyl,” as used herein, refers to a mono, bicyclic or tricyclic carbocyclic radical (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1 or 2 double bonds. Cycloalkyl groups may be unsubstituted or substituted by one or more suitable substituents, preferably 1 to 5 suitable substituents, as defined above.

The term “halo” or “halogen,” as used herein, refers to a fluoro, chloro, bromo or iodo radical.

The term “heteroaryl,” as used herein, refers to a monocyclic, bicyclic, or tricyclic aromatic heterocyclic group containing one or more heteroatoms (e.g., 1 to 3 heteroatoms) selected from O, S and N in the ring(s). Heteroaryl groups include, but are not limited to, pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, and indolyl. Heteroaryl groups may be unsubstituted or substituted by one or more suitable substituents, preferably 1 to 5 suitable substituents, as defined above.

The term “heterocycle” or “heterocyclyl,” as used herein, refers to a monocyclic, bicyclic, or tricyclic group containing 1 to 4 heteroatoms selected from N, O, S(O)ₙ, P(O)ₙ, PR_{z}, NH or NR_{z}, wherein R_{z} is a suitable substituent. Heterocyclic groups optionally contain 1 or 2 double bonds. Heterocyclic groups include, but are not limited to, azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, and benzoxazinyl. Examples of monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholin-yl, 1,2-tetrahydrothiazin-2-yl, 1,3-tetrahydrothiazin-3-yl, tetrahydrothiadiazin-yl, morpholin-yl, 1,2-tetrahydrodiazin-2-yl, 1,3-tetrahydrodiazin-1-yl, 1,4-oxazin-2-yl, and 1,2,5-oxathiazin-4-yl. Heterocyclic groups may be unsubstituted or substituted by one or more suitable substituents, preferably 1 to 3 suitable substituents, as defined above.

The term “hydroxy,” as used herein, refers to an —OH group.

The term “suitable substituent,” as used herein, is intended to mean a chemically acceptable functional group, preferably a moiety that does not negate the activity of the inventive compounds. Such suitable substituents include, but are not limited to halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxycarbonyl groups, alkylsulfonyl groups, and arylsulfonyl groups. Those skilled in the art will appreciate that many substituents can be substituted by additional substituents.

The term “water cut,” as used herein, means the percentage of water in a composition containing an oil and water mixture.

Having described the invention in detail, it will be apparent that modifications and variations are possible without departing from the scope of the invention defined in the appended claims.

### EXAMPLES

The following non-limiting examples are provided to further illustrate the invention.

### Example 1: General Synthesis of Compounds

### Step 1: Synthesis of ASA-Polyamine as an Intermediate

An ASA-polyamine intermediate (III) is first obtained by an imidation reaction between a diamine or polyamine (I) and alkenyl succinic anhydride (II) as depicted in Scheme 1.

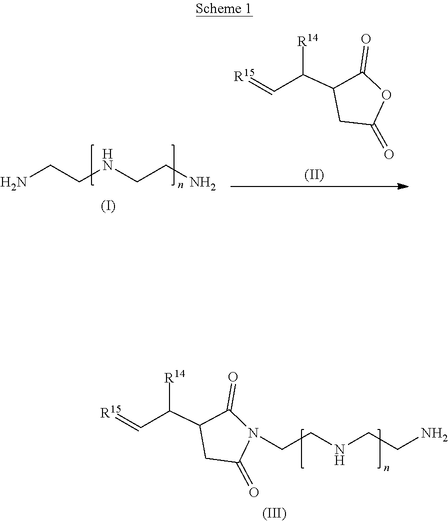

Wherein, n=1 to 100

### Step 2: Michael Addition Reaction Between ASA-Polyamine Intermediate and Cationic/Anionic Monomer

The second step involves an aza-Michael addition reaction between intermediate (III) and an α,β-unsaturated carbonyl compound containing at least one polar (charged) group (NR¹¹R¹²R¹³(+) X(−), —COOH, —SO₃H, —PO₃H, or a salt thereof) (IV) to afford multi-ionic compounds (V) as shown in Scheme 2.

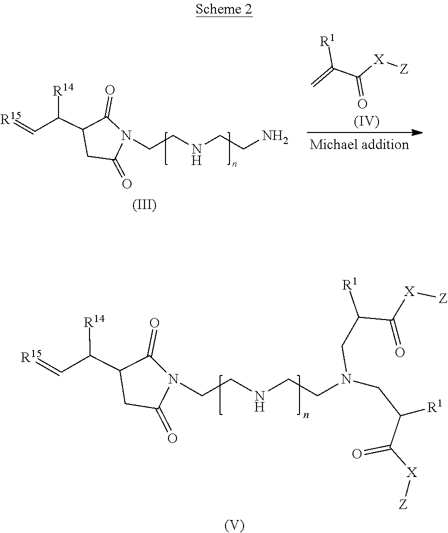

The generic synthesis reaction scheme for preparation of disclosed compounds using branched polyethyleneimine is shown in Scheme 3. The structure VI depicted below are a depiction of generalized reaction products.

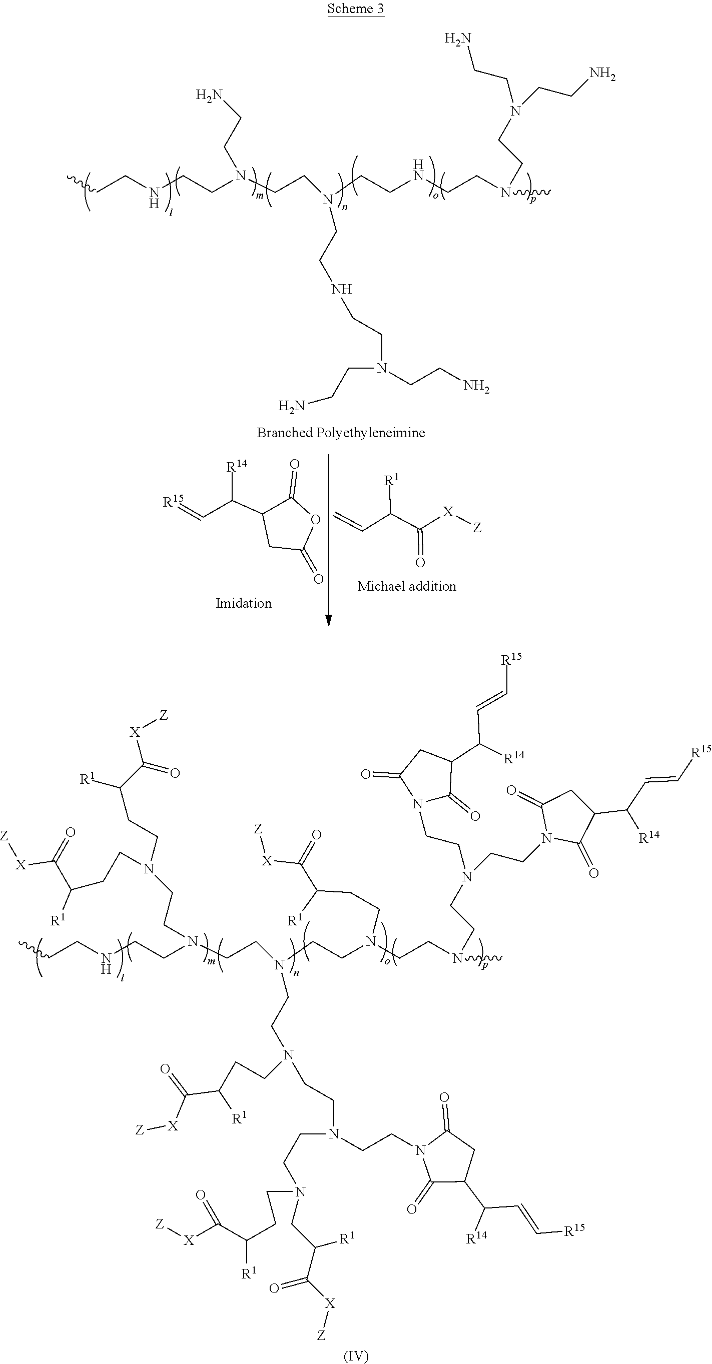

wherein:
k, l, m, m, o, p are an integer of 1-100,
X is NH or O,
R¹ is H, CH₃, or an unsubstituted, linear or branched C₂-C₁ alkyl group,
R¹⁴ and R¹⁵ are independently C₆ to C₁₂ alkyl;
Z is hydrogen, L¹-NR¹¹R¹²R¹³, L²-PR¹¹R¹²R¹³, L³-COOH, L⁴-SO₃H, L⁵-PO₃H, or a salt thereof;
L¹, L², L³, L⁴, and L⁵ are independently C₁-C₁₀ alkylene or alkenylene;
R¹¹, R¹², and R¹³ are independently C₁-C₁ alkyl group or benzyl group.

### Example 2: Specific Multi-Ionic Surfactant Synthesis

Following the synthetic approach described in Schemes 1-3 in earlier section, multi-ionic surfactants were synthesized by varying ionic monomer type and molar ratios.

TABLE 3

| | | | | Molar ratio |
|---|---|---|---|---|
| | | | | (ASA:Amine:Ionic |
| # | ASA | Amine | Monomer | monomer) |
| 1. | C18-ASA | Pentaethylenehex | (3-Acrylamidopropyl) | 1:1:4 |
| | | amine (PEHA) | trimethylammonium chloride | |
| | | | (APTAC) | |
| 2. | C-16 | PEHA | APTAC | 1:1:4 |
| | ASA | | | |
| 3. | C18-ASA | PEHA | APTAC | 1:1:3 |
| 4. | C-16 | PEHA | APTAC | 1:1:3 |
| | ASA | | | |
| 5. | C18-ASA | PEHA | APTAC | 1:1:5 |
| 6. | C18-ASA | PEHA | 2-Acrylamido-2-methyl-1- | 1:1:2 |
| | | | propanesulfonic acid sodium | |
| | | | salt (NaAMPS) | |
| 7. | C16-ASA | PEHA | NaAMPS | 1:1:2 |

Synthesis of one representative example (compound 1 in Table 3) of disclosed multi-cationic compound is described below in Scheme 4.

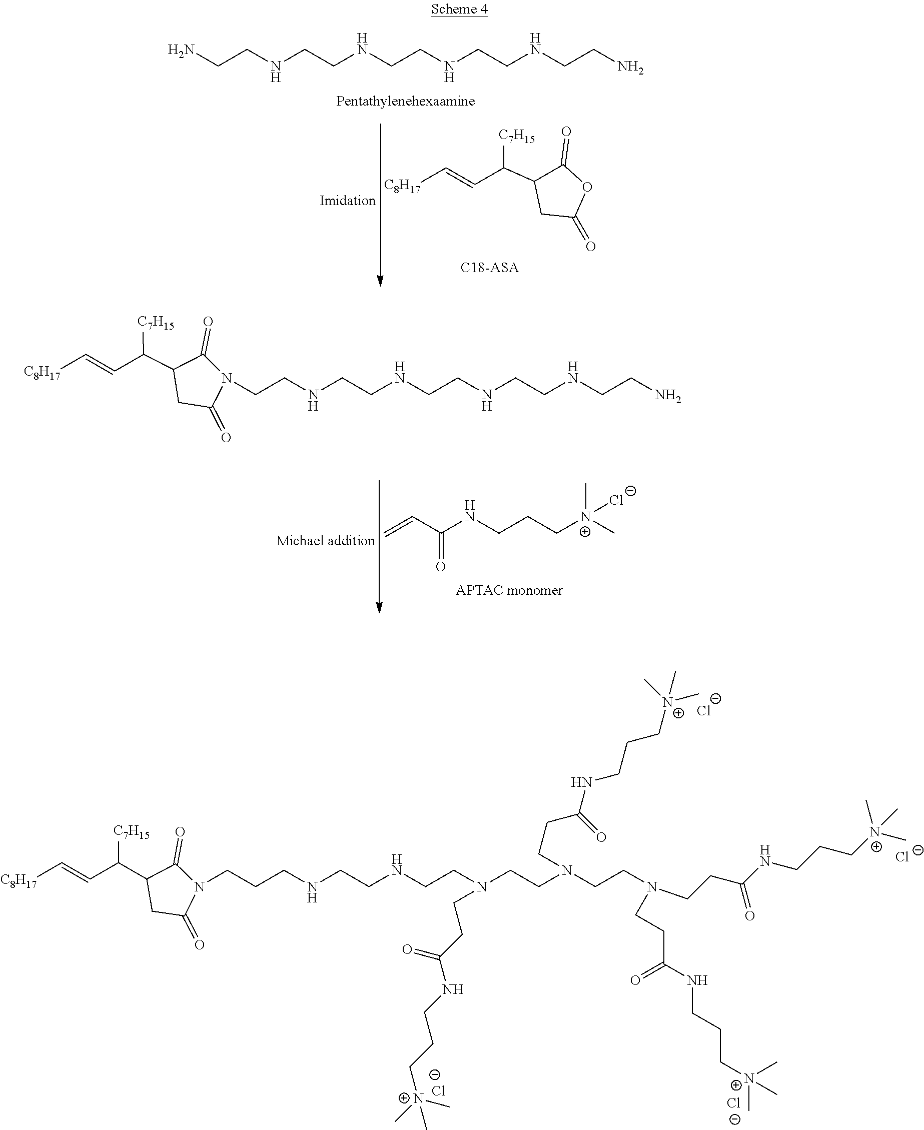

Step 1 (Imidation): Procedure: To a 250 mL three-necked round-bottom flask equipped with a temperature probe, nitrogen inlet, Dean-Stark apparatus, condenser and magnetic stir bar was added i-octadecenylsuccinic anhydride (iODSA, 98%, 120 g, 0.33 eq). Pentaethylenehexamine (PEHA, 78 g, 0.3 eq) was then charged to the well-stirred reaction mixture in a 1:1 ASA/polyamine molar ratio. The resulting mixture was heated gradually to 120° C. with nitrogen blanketing, then heated up to 150° C. with nitrogen purging, and stirred at 150° C. with nitrogen purging for 3 to 5 hours or until completion of the reaction to result in the desired product.
Step 2 (Michael addition reaction): To a 250 mL three-necked round-bottom flask equipped with a temperature probe, nitrogen inlet, condenser and magnetic stir bar was added ASA-polyamine intermediate (45 g) obtained from step 1. The acrylamidopropyl) trimethylammonium chloride (APTAC, 75% aqueous solution, 86 g) was then charged to the well-stirred reaction mixture in a 1:4 molar ratio. The resulting mixture was stirred to 80° C. overnight or until completion of reaction.

Similarly, another representative example of polyhydroxy anionic compounds is shown below:

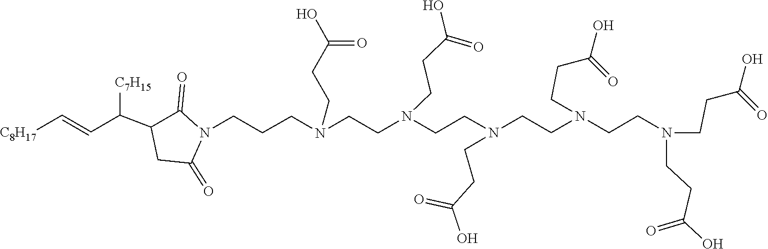

Another example of multi-ionic surfactants with two hydrophobic tails is shown below:

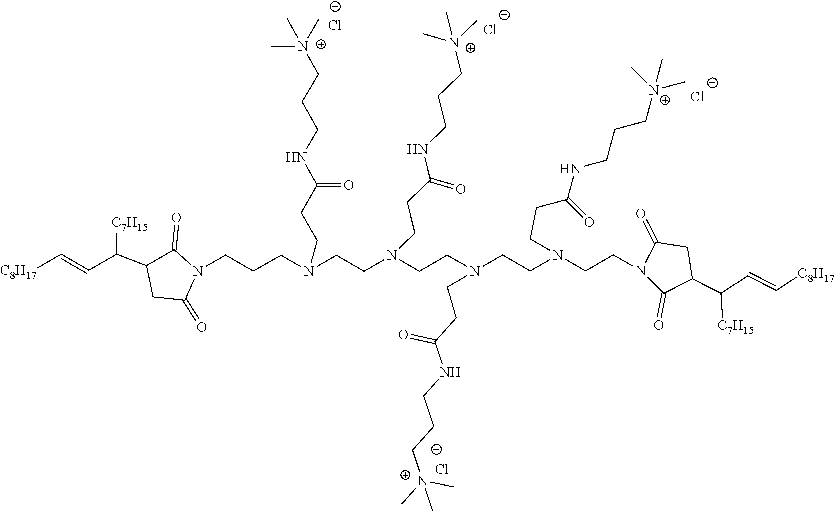

The terms “comprise(s),” “include(s),” “having,” “has,” “can,” “contain(s),” and variants thereof, as used herein, are intended to be open-ended transitional phrases, terms, or words that do not preclude the possibility of additional steps or components. The singular forms “a,” “and,” “the” and “said” include plural references unless the context clearly dictates otherwise. The present disclosure also contemplates other embodiments “comprising,” “consisting of” and “consisting essentially of,” the embodiments or elements presented herein, whether explicitly set forth or not.

In view of the above, it will be seen that the several objects of the invention are achieved and other advantageous results attained.

As various changes could be made in the above compositions and processes without departing from the scope of the invention, it is intended that all matter contained in the above description shall be interpreted as illustrative and not in a limiting sense.

1. A multi-ionic compound having the structure of Formula 1 corresponding to: 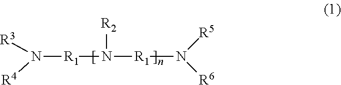
wherein
R¹ is independently C₁-C₁₀ alkylene;
R² is independently hydrogen, —(CH₂)ₓ—NR²⁰R²¹, or —(CH₂)ₓ—C(R¹⁰)—C(O)—X—Z;
R³ and R⁴ are independently hydrogen, —(CH₂)ₓ—C(R¹⁰)—C(O)—X—Z, or R³ and R⁴ together with the nitrogen they are attached to form a substituted nitrogen-containing heterocyclyl;
R⁵ and R⁶ are independently hydrogen, —(CH₂)ₓ—C(R¹⁰)—C(O)—X—Z, or R⁵ and R⁶ together with the nitrogen they are attached to form a substituted nitrogen-containing heterocyclyl;
R¹⁰ is independently hydrogen, alkyl, aryl, or alkaryl;
R¹¹, R¹², and R¹³ are independently C₁ to C₁₀ alkyl or alkaryl;
R²⁰ and R²¹ are independently hydrogen, —(CH₂)ₓ—NR²⁰R²¹, —(CH₂)ₓ—C(R¹⁰)—C(O)—X—Z, or together with the nitrogen they are attached to form a substituted nitrogen-containing heterocyclyl;
X is independently NH or O;
Z is independently hydrogen, L¹-NR¹¹R¹²R¹³, L²-PR¹¹R¹²R¹³, L³-COOH, L⁴-SO₃H, L⁵-PO₃H, or a salt thereof;
L¹, L², L³, L⁴, and L⁵ are independently C₁-C₁₀ alkylene or alkenylene;
n is an integer of 1 to 10; and
x is independently an integer of 1 to 10;
wherein at least one of R³ and R⁴, or R⁵ and R⁶ together form a substituted nitrogen-containing heterocyclyl; and
wherein at least one R² is —(CH₂)ₓ—C(R¹⁰)—C(O)—X—Z or —(CH₂)ₓ—NR²⁰R²¹ wherein one of R²⁰ or R²¹ is —(CH₂)ₓ—C(R¹⁰)—C(O)—X—Z.

2. (canceled)

3. The multi-ionic compound of claim 1, wherein R¹ is independently C₂-C₄ alkylene.

4. (canceled)

5. The multi-ionic compound of claim 3, wherein at least one R² is —(CH₂)ₓ—NR²⁰R²¹, or —(CH₂)ₓ—C(R¹⁰)—C(O)—X—Z.

6.-7. (canceled)

8. The multi-ionic compound of claim 5, wherein x is independently 1 to 3.

9. (canceled)

10. The multi-ionic compound of claim 1, wherein R³ and R⁴ together with the nitrogen they are attached to form: 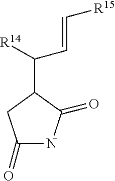
wherein R¹⁴ is a C₆ to C₁₂ alkyl and R¹⁵ is a C₆ to C₁₂ alkyl.

11. (canceled)

12. The multi-ionic compound of claim 1, wherein R⁵ and R⁶ together with the nitrogen they are attached to form: 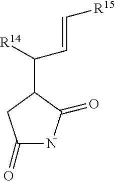
wherein R¹⁴ is a C₆ to C₁₂ alkyl and R¹⁵ is a C₆ to C₁₂ alkyl.

13. (canceled)

14. The multi-ionic compound of claim 1, wherein R¹⁰ is hydrogen.

15. The multi-ionic compound of claim 1, wherein X is O.

16. The multi-ionic compound of claim 15, wherein Z is independently hydrogen or L³-COOH, L⁴-SO₃H, L⁵-PO₃H, or a salt thereof; and L³, L⁴, and L⁵ are independently C₁ to C₆ alkylene.

17. (canceled)

18. The multi-ionic compound of claim 15, wherein Z is hydrogen.

19. The multi-ionic compound of claim 1, wherein X is NH.

20. The multi-ionic compound of claim 19, wherein Z is independently L¹-NR¹¹R¹²R¹³, L²-PR¹¹R¹²R¹³, L³-COOH, L⁴-SO₃H, L⁵-PO₃H, or a salt thereof; and L³, L⁴, and L⁵ are independently C₂ to C₃ alkylene.

21.-24. (canceled)

25. The multi-ionic compound of claim 20, wherein Z is L¹-NR¹¹R¹²R¹³ and R¹¹, R¹², and R¹³ are methyl.

26.-28. (canceled)

29. A method of treating an aqueous medium, the method comprising contacting the aqueous medium with the multi-ionic compound of claim 1.

30. A fabric softening composition comprising the multi-ionic compound of claim 1.

31. A method of softening fabrics comprising contacting the fabric with an effective amount of the multi-ionic compound of claim 1.

32. A method of inhibiting corrosion in a system comprising contacting the fluid in the system with an effective amount of the multi-ionic compound of claim 1.

33. A method of inhibiting biofilm growth in a system comprising contacting the fluid in the system with an effective amount of the multi-ionic compound of claim 1.

34. A method of inhibiting bacterial growth in a system comprising contacting the fluid in the system with an effective amount of the multi-ionic compound of claim 1.

35. A method of affecting the rheology of a composition comprising contacting the composition with an effective amount of the multi-ionic compound of claim 1.
